(19) United States
(12) Patent Application Publication
(10) Pub. No.: US 2004/0087580 A1
(43) Pub. Date: May 6, 2004

(54) 4-Hydroxy-2H-thieno[2,3-e]-1,2-thiazine-3-carboxamide-1,1-dioxides, process for preparing and pharmaceutical compositions thereof

(75) Inventors: Guenter Trummlitz, Warthausen (DE); Wolfhard Engel, Biberach (DE); Wolfgang Eberlein, Biberach (DE); Guenther Engelhardt, Biberach (DE); Joanne Van Ryn, Warthausen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/624,145

(22) Filed: Jul. 21, 2003

Related U.S. Application Data
(60) Provisional application No. 60/408,147, filed on Sep. 4, 2002.

(30) Foreign Application Priority Data
Jul. 26, 2002 (EP) 02016686

(51) Int. Cl. A61K 31/549; C07D 513/00

(52) U.S. Cl. 514/224.2; 544/48

(57) ABSTRACT

The present invention relates to new 4-hydroxy-2H-thieno[2,3-e]-1,2-thiazine-3-carboxamide-1,1-dioxides of general formula

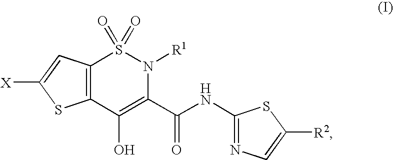

wherein X, is fluorine, chlorine, bromide or CF₃, R¹ is hydrogen or C₁-C₂ and R² is C₁-C₂, which compounds have, inter alia, anti-inflammatory activity.

This application is a non-provisional application of prior provisional application U.S. Ser. No. 60/408,147, filed Sep. 4, 2002, and also claims priority from EPA 02 016 686.4 filed Jul. 26, 2002, both of which are hereby incorporated by reference.

The invention relates to new 4-hydroxy-2H-thieno[2,3-e]-1,2-thiazine-3-carboxamide-1,1-dioxides of general formula

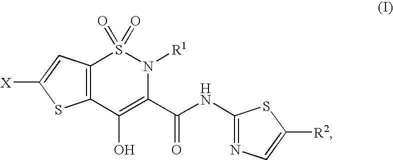

the physiologically acceptable salts thereof with inorganic or organic bases, processes for the preparation thereof and pharmaceutical compositions containing them.

In the above general formula I

X denotes a fluorine, chlorine or bromine atom or the trifluoromethyl group,

R¹ denotes a hydrogen atom or a methyl or ethyl group and

R² denotes a methyl or ethyl group.

### BACKGROUND OF THE INVENTION

Prostaglandins are formed from arachidonic acid and play an important role in inflammatory processes. As a result, the inhibition of prostaglandin production, especially the production of PGH₂, is a current target of research into anti-inflammatory substances.

Non-steroidal anti-inflammatory active substances (NSAIDs) block prostaglandin—synthesis by inhibiting the enzyme cyclooxygenase. Conventional NSAIDs not only bring about a reduction in the pain and swelling which accompany inflammatory processes but also actively influence other prostaglandin-regulated processes which are not connected with the inflammatory process. Therefore, high doses of most conventional NSAIDs involve various side effects, including life-threatening ulcers in the gastro-intestinal tract, which substantially reduce their therapeutic potential.

In 1971, J. R. Vane (Nat. New Biol. 1971, 231, 232-235) was able to show that the activity of the NSAIDs is based on their ability to inhibit the activity of cyclooxygenase (COX), leading to a reduced synthesis of proinflammatory prostaglandins.

The enzyme cyclooxygenase (COX) catalyses the first step of the synthesis of prostanoids and exists in the form of two different isoenzymes: the constitutively expressed isoform COX-1 and the isoform COX-2 which is induced in the course of an inflammation (J. Y. Fu et al., J. Biol. Chem. 1990, 265, 16737-16740). COX-1 is formed in virtually all tissues and mediates physiological responses (e.g. protection of the cells of the stomach, thrombocyte aggregation). COX-2 is formed by cells which are involved in inflammatory processes (e.g. macrophages, monocytes, synoviocytes), and is primarily responsible for the synthesis of the prostanoids which are involved in pathological processes such as acute or chronic inflammatory conditions.

Whereas the inhibition of the COX-1 activity by NSAIDs leads to a number of side effects, such as e.g. gastrointestinal ulcers and bleeding or thrombocyte dysfunction, the inhibition of the prostanoids formed by COX-2 produces the anti-inflammatory, analgesic and antipyretic activity of the NSAIDs. The hypothesis arising from this, that selective COX-2 inhibition could have similar therapeutic effects to those of the NSAIDs, but without the side effects mentioned earlier, was the basis for the development of selective COX-2 inhibitors as a new class of anti-inflammatory and analgesic substances with improved gastrointestinal tolerance.

Structurally, the new substances belong to the class of the enolcarboxamides (oxicams). One substance belonging to this class which has recently come on the market is lornoxicam, but this does not have a COX-2-selective activity. The inhibitory potential of lornoxicam was tested in two largely identical assays in which different types of human cells were used which are specific for COX-1 and COX-2. Lornoxicam inhibited the formation of TXB₂ in HEL cells in dose-dependent manner and the formation of PGF_{1α} in LPS-stimulated Mono Mac 6 cells. The two COX-isoenzymes were largely inhibited to the same extent, with IC₅₀ values of 0.003 μM for COX-1 and 0.008 μM for COX-2 (J. Berg et al., Inflamm. Res. 1999, 48, 369-379).

Most of the selective COX-2-inhibitors currently on the market belong to the class of the diarylheterocycles (coxibs) (Review: A. S. Kalgutkar, Z. Zhao, Current Drug Targets 2001, 2, 79-106). The mechanism of the COX-2 selectivity of this category of substances is based on the irreversible inhibition of the COX-2 activity, whereas the COX-1 activity is inhibited reversibly. As a result, the inhibitory concentrations for COX-2 are substantially lower than for COX-1. This also means that substances of this class inhibit the COX-2 activity for a longer time, even in the case of shorter plasma half lives. Thus, valdecoxib, first described in U.S. Pat. No. 5,633,272, has a plasma half-life of about 8 hours, but acts for more than 24 hours, as the COX-2 activity is irreversibly inhibited (Valdecoxib Product Information).

One possible cause of the increased risk of myocardial infarct during treatment with coxibs, e.g. with rofecoxib (VIGOR Study, C. Bombardier et al. N. Eng. J. Med. 2000, 343, 1520-1528), may be found in the high and irreversible inhibition of the COX-2 activity, with at the same time no effect whatever on the COX-1 activity in the therapeutic dosage range. This leads to an imbalance of PGI₂ and TXA₂.

It is now the object of the present invention to provide new COX-2 selective anti-inflammatory, analgesic and antiphlogistic substances which are characterised by a rapid onset of activity and a short half-life and may thus preferably be used in acute pain therapy, but also in chronic pain relief. The substances should also have a low distribution volume in the body and reversibly block the COX-2 receptors.

### DETAILED DESCRIPTION OF THE INVENTION

Surprisingly it has been found that the compounds of general formula I solve the problem described above and have outstanding antiphlogistic activity, inhibit the pain of inflammation and are particularly suitable for treating rheumatic diseases.

The compounds according to the invention are therefore suitable for treating all peracute, acute, subacute, chronic and recurring inflammation, particularly for treating the symptoms of acute episodes of intermittent or chronic activated arthrosis as well as for long-term symptomatic treatment of rheumatoid arthritis (chronic polyarthritis) and for the symptomatic treatment of ankylosing spondylitis (Bechterew's disease).

It has also been found that the compounds of general formula I are suitable for the prevention and treatment of neoplasias which produce prostaglandins or secrete cyclooxygenase, including benign and cancerous tumours, growth disorders and polyps.

Neoplasias which (frequently) produce prostaglandins comprise for example malignant brain tumours, bone cancer, epithelial cell neoplasia such as basal cell carcinoma, adenocarcinoma, cancers of the gastrointestinal tract such as lip cancer, mouth cancer, oesophageal cancer, cancer of the small intestine and stomach cancer, large bowel cancer, liver cancer, bladder cancer, pancreatic cancer, ovarian cancer, cancer of the womb, lung cancer, breast cancer and skin cancer, prostate cancer, kidney cell carcinoma and other known types of cancer which affect the epithelial cells in the body.

The compounds according to the invention are also suitable for treating acute pain, such as for example toothache after tooth extractions, post-traumatic and postoperative pain, headache, acute sciatica, acute back pain, tendonitis, cervicobrachial syndrome and tennis elbow as well as for the treatment of persistent pain, such as for example backache or pain caused by tumours.

The new compounds of general formula I may be incorporated in the usual pharmaceutical formulations for use as medicaments. The single dose for adults is from 1 to 300 mg, preferably 5 to 50 mg, administered once to three times a day.

For therapy the compounds according to the invention may be given in monotherapy or in combination with substances with a vasomodulator effect, H₁-antagonists, leukotriene antagonists, iNO-synthase inhibitors, antithrombotics or substances which influence angiogenesis, simultaneously or gradually over time.

Substances with a vasomodulator effect which may be used include, according to the invention, renin angiotensin system antagonists, nitrovasodilators, direct vasodilators, calcium channel blockers, phosphodiesterase inhibitors, sympathomimetics, sympatholytics or iNOS inhibitors.

Examples of phosphodiesterase inhibitors include according to the invention the compounds selected from the group consisting of amrinone [5-amino-(3,4′-bipyridin)-5(1H)-one], milrinone [1,6-dihydro-2-methyl-6-oxo-(3,4′-bipyridin)-5-carbonitrile-lactate] and vesnarinone [3,4-dihydro-6-(4-(3,4-dimethoxybenzoyl)-1-piperazinyl)-2(1H)-quinoline].

H₁-antagonists which may be used according to the invention are those compounds which are selected from the group consisting of epinastine, cetirizine, azelastin, fexofenadin, levocabastin, loratadine, mizolastine, ketotifen, emedastine, dimetindene, clemastin, bamipine, cexchlorpheniramine, pheniramine, doxylamine, chlorophenoxamine, dimenhydrinate, diphenhydramine, promethazine, ebastine, desloratidine and meclozine, while a combination with epinastin is of particular importance according to the invention.

Leukotriene antagonists which may be used according to the invention are those compounds which are selected from the group consisting of leukotriene-B4-antagonists, including peptidic leukotriene-B4-modulators, as well as leukotriene-A4-hydrolase inhibitors. Examples of leukotriene-B4-antagonists are ebselen, PZ 51, DR 3305, HARMOKISANE, Biomed-101, S-2474, Ono 3403, DW-1141, SA-6541, S-9499, 2-aminopyrimidine derivatives, as disclosed in International Patent Application WO 97/24328, LY 292728, Moxilubant, CGS-25019C, Ono 4057 (LB-457), CP-195543 (Pfizer), LTB-019, Balazipone (OR-1364), RG-14893, ZK 158252, Ticolubant (SB-209 247), VML 295 (LY 293 111).

iNO-Synthase inhibitors which may be used according to the invention are particularly those compounds which are selected from the group consisting of:

1. derivatives of the compounds L-N⁶-(1-iminoethyl)-lysine and L-N⁵-(1-iminoethyl)-ornithine, such as for example 2-amino-6-(1-imino-2-fluoroethylamino)-4,4-dioxo-4-thiohexanoic acid.

2. non-amino acid inhibitors, preferably isothioureas, and also inhibitors of other types of compounds such as amidines, guanidines, phenylpyrimidines, indazoles, phenylimidazoles or phencyclidines.

According to the invention, S-ethyl-thiourea, 2-amino-5,6-dihydro-6-methyl-4H-1,3-thiazine, AC-C102222 or GW 273629 may preferably be used, for example.

Suitable antithrombotics which may be used according to the invention include, in particular, those compounds which are selected from the group consisting of inhibitors of blood platelet aggregation, such as for example aspirin, inhibitors of thromboxane synthesis, thromboxane receptor antagonists, thienopyridine or glucoprotein receptor antagonists.

Thromboxane receptor antagonists belong to various chemical structural categories, while derivatives of 7-oxabicycloheptane-oxazole, such as for example SQ 33,961 or BMS-180291, have proved particularly satisfactory.

Among the inhibitors of thromboxane synthesis the dual thromboxane synthesis inhibitors/thromboxane receptor antagonists such as isbogrel, ridogrel and terbogrel, but also various sulphonamide compounds such as CGS 22652 are of particular interest according to the invention.

Of the thienopyridines, ticlopidine, clopidogrel or PCR-4009 are of particular interest according to the invention.

Examples of glucoprotein receptor antagonists which may be used include both peptidic and peptidomimetic antagonists, such as for example benzamidine derivatives.

Examples of substances which influence angiogenesis include, according to the invention, in particular the compounds which are selected from the group consisting of: proteins and inhibitors with a high molecular weight, such as for example the vascular permeability factors (VPF), the vascular endothelial growth factors (VEGF), the fibroblast growth factors (bFGF, aFGF) or endostatin as well as inhibitors of various types of substances with a low molecular weight, such as for example inhibitors of urokinase-plasminogen activators and matrix metalloproteinases, such as for example batimastat or marimastat. In addition it is also possible according to the invention to use fumagillin and derivatives thereof, such as for example FR-118487.

The abovementioned compounds of general formula I may be obtained by the following methods:

(a) All the compounds of general formula I may be obtained by reacting 4-hydroxy-2H-thieno[2,3-e]-1,2-thiazine-3-carboxylic acid ester-1,1-dioxides of general formula

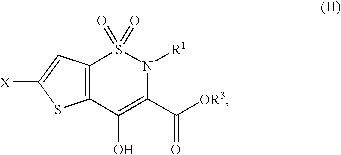

wherein

X and R¹ are as hereinbefore defined and R³ denotes a C₁₋₈-alkyl group, an arylalkyl group with 7 to 10 carbon atoms or a phenyl group, with a 2-thiazolamine of general formula

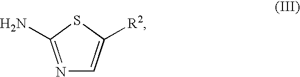

substituted in the 5 position,

wherein

R² is as hereinbefore defined.

The reaction of the carboxylic acid esters of general formula II with the 2-thiazolamines of general formula III is carried out in suitable, inert organic solvents, for example in aromatic hydrocarbons such as benzene, toluene, xylene, chlorobenzene, o-dichlorobenzene or tetrahydronaphthalene, in dimethylformamide, dimethylacetamide or dimethylsulphoxide, in ethers such as dimethoxyethane, diethyleneglycol dimethyl ether or diphenylether or also directly in the excess amine. The work is done at a temperature of 60 to 200° C. The reaction preferably takes place in toluene or xylene at boiling temperature and the alcohol formed in the reaction is eliminated by azeotropic distillation or by refluxing, for example using a Soxhlet extractor charged with molecular sieves. The product crystallises out directly from the reaction mixture or is precipitated by the addition of water when using a water-miscible solvent.

(b) Compounds of general formula I wherein X and R² are as hereinbefore defined and R¹ denotes a methyl or ethyl group may also be obtained by reacting a 4-hydroxy-2H-thieno[2,3-e]-1,2-thiazine-3-carboxamide-1,1-dioxide of general formula

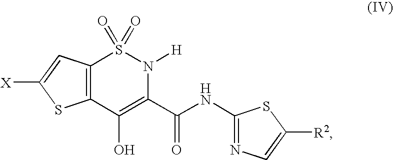

wherein

X and R² are as hereinbefore defined, with an alkylating agent of general formula

R¹¹—Y, (V)

wherein

Y denotes a leaving group such as a halogen atom or the group —OSO₂R¹², R¹¹ denotes a methyl or ethyl group and R¹² denotes an alkyl, aryl or arylalkyl group or the trifluoromethyl group, in the presence of bases.

Suitable bases may be alkali or alkaline earth metal hydroxides, for example sodium, potassium or barium hydroxide, or alkali or alkaline earth metal carbonates, such as for example sodium or potassium carbonate, as well as alkali or alkaline earth metal alkoxides, for example sodium methoxide, potassium methoxide, potassium-tert.-butoxide, or tertiary amines, for example triethylamine, if the work is done in an aqueous medium, in an alcoholic medium, such as for example in methanol, ethanol, n-propanol, 2-propanol or in mixtures of these solvents.

The alkylating agent, preferably methyl or ethyl bromide or iodide, dimethyl-sulphate or diethylsulphate, is added directly in the form of a solution in a suitable solvent to the other components in the reaction mixture, while in the case of methyl bromide the work is done in a closed apparatus. As further solvents it is possible to use dimethylformamide, dimethylacetamide, dimethylsulphoxide, hexa-methylphosphoric acid triamide or sulpholane.

If alkali or alkaline earth metal carbonates are used as bases, aliphatic ketones such as acetone, for example, may also be used as solvent.

If the reaction is carried out in aprotic organic solvents, e.g. in dimethylformamide, dimethylsulphoxide, tetrahydrofuran or another open-chained or cyclic ether, alkali metal hydrides or alkaline earth metal hydrides, for example sodium hydride, may also be used as bases. However, the alkylating agent of general formula V is not added until the alkali metal hydride or alkaline earth metal hydride has finished reacting with the starting compound of general formula IV. The reaction temperature is +20 to +120° C.

(c) All the compounds of general formula I may be obtained by reacting 4-hydroxy-2H-thieno[2,3-e]-1,2-thiazine-3-carboxamide-1,1-dioxides of general formula

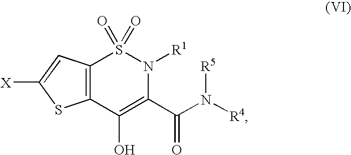

wherein

X and R¹ are as hereinbefore defined and R⁴ and R⁵, which may be identical or different, represent a hydrogen atom, a C₁₋₈-alkyl group, a C₃₋₁₀-cycloalkyl group, an arylalkyl group with 7 to 10 carbon atoms or a phenyl group optionally substituted by nitro, alkoxy or alkyl groups or together with enclosed nitrogen atom denote a 1-azetidinyl, 1-pyrrolidinyl, 1-piperidinyl, hexahydro-1H-1-azepinyl or octahydro-1-azocinyl group,

with a 2-thiazolamine of general formula III.

The reaction of the carboxylic acid amide of general formula VI with 2-thiazolamines of general formula III takes place in suitable, inert organic solvents, for example in aromatic hydrocarbons such as e.g. benzene, toluene, xylene or o-dichlorobenzene, in dimethylformamide, dimethylacetamide, dimethylsulphoxide or in hexamethylphosphoric acid triamide, in ethers such as e.g. dimethoxyethane, diethyleneglycol dimethylether or diphenylether, or also directly in the excess amine. The work is done at temperatures between 80 and 200° C. Preferably, the reaction is carried out in xylene at boiling temperature, catalytic amounts of p-toluenesulphonic acid are added and the aromatic amine is used in excess. The product either crystallises out directly from the reaction mixture or is obtained by evaporating the solvent. However, it may also be precipitated by the addition of water, if a water-miscible solvent is used.

The compounds of general formula I may, if desired, be converted into the physiologically acceptable salts thereof with inorganic or organic bases by methods known per se. Suitable bases include, for example, alkali metal alkoxides, alkali metal hydroxides, alkaline earth metal hydroxides, trialkylammonium hydroxides or alkylamines.

The esters of general formula II used as starting compounds are known from the literature and may be prepared for example according to the instructions contained in EP-A-0 001 113.

The compounds of general formula III are also known from the literature (cf. H. Erlenmeyer, Z. Herzfeld and B. Prijs, Helv. Chim. Acta 1955, 38, 1291; K. D. Kulkarni and M. V. Shirsat, J. Sci. and Ind. Research (India) 1959, 18B, 411; C. A. 1960, 54, 14230 d).

The starting compounds of general formula IV are prepared from 4-hydroxy-2H-thieno[2,3-e]-1,2-thiazin-3-carboxylic acidester-1,1-dioxides of general formula II wherein R¹ denotes a hydrogen atom, by reacting with 2-thiazolamines of general formula III substituted in the 5 position in suitable, inert organic solvents at temperatures between 60 and 200° C.

The starting compounds of general formula VI may be prepared, completely analogously to process (a) above, from the 4-hydroxy-2H-thieno[2,3-e]-1,2-thiazin-3-carboxylic acidester-1,1-dioxides of general formula II by reacting with amines of general formula R⁴NH₂ wherein R⁴ is as hereinbefore defined, in an inert solvent at temperatures between 60 and 200° C. while constantly eliminating the alcohol thus released by azeotropic distillation.

The invention further relates to pharmaceutical compositions which contain the compounds of general formula I or the physiologically acceptable salts thereof with inorganic or organic bases, for example plain or coated tablets, capsules or suppositories.

As already mentioned hereinbefore the 4-hydroxy-2H-thieno-[2,3-e]-1,2-thiazine-3-carboxamide-1,1-dioxides of general formula I and the physiologically acceptable salts thereof with inorganic or organic bases have valuable pharmacological properties. These compounds have a powerful anti-inflammatory effect, they relieve the pain of inflammation and are particularly suitable for treating rheumatic diseases.

For example, the substance

A=6-chloro-4-hydroxy-2-methyl-N-(5-methyl-2-thiazolyl)-2H-thieno[2,3-e]-1,2-thiazine-3-carboxamide-1,1-dioxide

was investigated in the rat after oral administration for its antiphlogistic activity using the kaolin and carrageenin oedema test.

Test 1: Carageenin Oedema Assay

The effect on kaolin-induced oedema in the hind paw of the rat was tested on male Chbb:Thom rats weighing between 120 and 145 g. The kaolin oedema was induced according to the method described by Hillebrecht (Arzneimittel-Forsch. 1954, 4, 607) by a subplantar injection of 0.05 ml of a 10% suspension of kaolin in 0.85% saline solution into a hind paw. The other hind paw was given the same volume of 0.85% saline solution by subplantar route. The thickness of the paw was measured as described by Doepfner and Cerletti (Int. Arch. Allergy appll. Immunol. 1958, 12, 89) by determining the maximum sagittal diameter using indicating callipers with a constant contact pressure before and 5 hours after the induction of the oedema.

The test substances were administered 30 minutes before the induction of the oedema as a trituration in 1% tylose (1 ml/100 mg/animal) by oesophageal tube.

The diameter of the paw measured at the start of the test and the increase in diameter caused by the injection, read off on the adjacent paw, were subtracted from the diameter of the kaolin-treated paw and the difference was further calculated as the true threshold value.

The ED₃₅ for the kaolin oedema was calculated, after linear regression analysis with the confidence limits according to Fieller (Quart. J. Pharm. Pharmacol. 1944, 17, 117), as the dose which leads to a 35% reduction in the swelling of the paw compared with that observed in the control animals.

The carrageenin oedema was induced as described by Winter et al. (Proc. Soc. Exp. Biol. Med. 1962, 111, 544) by subplantar injection of 0.05 ml of a 1% solution of carrageenin in 0.85% sodium chloride solution. The test substances were administered 3 hours before the induction of the oedema. To evaluate the oedema-inhibiting effect the measurement obtained 60 minutes after induction of the oedema was used. The other details of the test method and the evaluation of the results are as described above for the kaolin oedema.

The effectiveness of compound A according to the invention in reducing swelling, measured as the ED35 by comparison with meloxicam, was 5.7 mg/kg as against 4.2 mg/kg in this experimental model.

Test 2:

The plasma half-life can be approximately determined indirectly by measuring the drop in the analgesic effect to half the maximum effect as a function of time, as described by Engelhardt et al. (Engelhardt et al., Inflamm. Res. 1995, 44, 423-433), while a modified method is used by Randall and Selitto (L. O. Randall, J. J. Selitto, Arch. Int. Pharmacodyn. 1957, 111, 409-419).

Here, male rats anaesthetised with ether and weighing from 100 to 150 g are injected with 0.1 mL of a freshly prepared suspension of 1.12 g of freeze-dried yeast in 18.9 mL of 5.55% glucose solution by subplantar injection into the right hind paw. Three hours after the subplantar administration of the yeast suspension the pain threshold is measured in grams of contact pressure. Then the animals are each given the test substances by oral route, 1.5, 3, 6 and 18 hours before the measurement of pain. An ED150 (e.g. the dosage which increases the pain threshold by 50%) is determined by linear regression analysis.

Test 3: Microsomal Assay:

The different degrees of inhibition of COX-1 and COX-2 can be determined using cell and cell-free tests in vitro. One example is microsomal assay (Inflammopharmacology 1996, 4, 125-135). This consists for example of insect cells which have been transfected with human COX-1 and COX-2 genes (for example using the Baculovirus system with human COX-1 and COX-2 clones). The microsomes were prepared from insect cells 3 days after the infection. The cell sediments were resuspended in 0.1 mol/L Tris buffer (pH 7.4), containing EDTA (10 mmol/L), PMSF (1 mmol/L), leupeptin (2 μg/ml), soya bean trypsin inhibitor (2 μg/ml) and aprotinin (1 mmol/L). The cells were treated with ultrasound and centrifuged at 10000 g for 10 minutes. The supernatant was removed and re-centrifuged at 100000 g for 1.5 hours. The microsomal sediment was then resuspended in 0.1 mol/L Tris buffer (pH 7.4) containing 30% glycerol, and stored at −80° C.

The microsomal preparations were thawed as required to 3.5 and 0.4 μg of protein/source or for hCOX-1 and h-COX 2 and incubated with haematin (2 μmol/L), phenol (0.5 mmol/L) and reduced glutathione (1 mmol/L) for 5 minutes at ambient temperature.

The test compound or a suitable DMSO medium was then added and the mixture was pre-incubated for 20 minutes before the addition of 2 μmol/L arachidonic acid. After 20 minutes the reaction was stopped with 0.1 N hydrochloric acid. The samples were diluted with EIA buffer containing 25 μmol/L of indomethacin and investigated by EIA for PGE₂ (RPN222, extended range protocol, Amersham).

In the abovementioned assay, compound A according to the invention inhibited COX-1 with an IC₅₀ of 0.5 μM and inhibited COX-2 with an IC₅₀ of 0.05 μM (COX-2 selectivity ratio of 10). Meloxicam was used as the comparison substance and inhibited COX-1 with an IC₅₀ of 36 μM and inhibited COX-2 with an IC₅₀ of 0.49 (ratio 73.5).

Test 4: Whole Blood Assay:

Another example is the human whole blood assay which is carried out according to a method of Patrignani et al. (P. Patrignani, M. R. Panana, A. Greco, O. Fusco, C. Natoli, S. Iacobelli et al., J. Pharmacol. Exp. Ther. 1994, 271, 1705-1710). For this, at least five test subjects donate blood. None have taken any aspirin for at least 10 days before donating the blood.

Measurement of the COX-1 activity. Human venous blood (50 mL) is collected in a polypropylene test tube without any anticoagulants. 2 μL aliquots of the test substance are pipetted into each previously labelled test tube (in triplicate) before the blood is collected, so as to achieve the appropriate final concentration. Whole blood (500 μL) is immediately added to each test tube. All the test tubes are mixed thoroughly and placed in an incubator for 60 minutes at 37° C. (5% CO₂).

The assay is stopped by centrifuging the samples for 5 minutes at 12000 g. 100 μL of the serum are taken and mixed with 400 μL methanol. Then centrifuging is continued for another 5 minutes at 12000 g. The thromboxane B₂ (TxB₂) levels in the supernatant are determined using a Standard Assay Kit for human thromboxane B₂ (example: TxB₂ EIA System, Amersham Pharmacia, Freiburg, Germany).

Measurement of the COX-2 activity: Human venous blood (50 mL) is collected in a polypropylene test tube with the anticoagulant (heparin, 10 IU per mL of whole blood, final concentration) and mixed carefully. 2 μL aliquots of the test substance are pipetted into previously labelled test tubes (in triplicate) before the blood is collected, so as to achieve the appropriate final concentration. The anticoagulated blood (500 μL) is added to each test tube. This is mixed thoroughly and incubated for 15 minutes at 37° C. Lipopolysaccharides (LPS, 5 mg/mL solution) of the Escherischia Coli serotype 0111:B4 (Sigma, St. Louis, Mich., USA) are dissolved in phosphate-buffered saline. Then 10 μL of LPS (final concentration, 100 μL/mL) are added to each test tube to activate the monocytes in the whole blood and induce COX-2 expression. The test tubes are thoroughly mixed again and incubated for 24 hours at 37° C. Then the samples are centrifuged for 5 minutes at 1200 g to obtain the cell-free plasma. The plasma (100 μL) is mixed with 400 μL of methanol. The samples are centrifuged again for 5 minutes at 1200 g and the PGE₂ concentrations in the supernatant are determined using a Standard Assay Kit for human PGE₂ (example: PGE₂ EIA Kit, Assay Designs, Inc., Ann Arbor, Mich., USA).

In the human whole blood assay described above the following results were obtained: compound A according to the invention inhibited COX-1 with an IC₅₀ of 0.25 μM, and inhibited COX-2 with an IC₅₀ of 0.07 μM (COX-2 selectivity ratio of 3.6). Meloxicam inhibited COX-1 with an IC₅₀ of 4 μM and inhibited COX-2 with an IC₅₀ of 0.55 μM (ratio 7.3). Naproxen was used as a non-selective inhibitor and inhibited COX-1 with an IC₅₀ of 31 μM and inhibited COX-2 with an IC₅₀ of 91 μM (ratio 0.34).

All the substances are non-toxic in the doses which would be used for therapy.

Experimental Section

The Examples that follow are intended to illustrate the invention without restricting it:

### EXAMPLE 1

### 6-chloro-4-hydroxy-2-methyl-N-(5-methyl-2-thiazolyl)-2H-thieno[2,3-e]-1,2-thiazine-3-carboxamide-1,1-dioxide

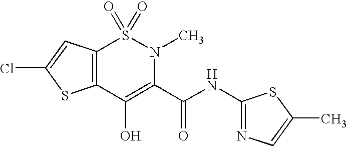

3.097 g (0.01 mol) methyl 6-chloro-4-hydroxy-2-methyl-2H-thieno-[2,3-e]-1,2-thiazin-3-carboxylate -1,1-dioxide and 1.25 g (0.011 mol) 5-methyl-2-thiazolamine are refluxed in 0.4 l of anhydrous xylene for 24 hours in a nitrogen atmosphere. The methanol formed is removed from the mixture using 4 Å molecular sieves which are contained in a Soxhlet apparatus. After the reaction has ended the mixture is left to cool to about 120° C., a spatula tip of animal charcoal is added and the mixture is briefly refluxed again. The reaction mixture at a temperature of about 120° C. is then filtered through a hot water funnel filled with glycerol and preheated to 120° C. The filtrate cooled to about 70° C. is triturated, whereupon orange crystals begin to be precipitated. The reaction mixture is left to stand overnight at a temperature of −5° C., the precipitate formed is suction filtered and washed thoroughly with ice-cold xylene. After recrystallisation from diisopropylether and 6 hours' drying in the vacuum drying cupboard at 1 mm Hg and 120° C., 2.64 g of product are obtained in the form of yellowish-orange crystals.

| | |
|---|---|
| Yield: | 67% of theory |
| Melting point: | T_{mp.} = 203-205° C. (decomposition) |
| Elemental analysis: | C₁₂H₁₀CIN₃O₄S₃ (391.86) |

| | | | | | |
|---|---|---|---|---|---|
| Calc.: | C 36.78 | H 2.57 | Cl 9.05 | N 10.72 | S 24.54 |
| Found: | C 36.95 | H 2.63 | Cl 8.95 | N 10.85 | S 24.47 |

IR (KBr): C═O 1615, amide-II 1520, SO₂ 1330, 1180/cm.

¹H-NMR (d₆-DMSO/CD₃OD; 400 MHz): δ/ppm=7.39 (1H-s: thiophene-H);

7.01(1H-q, J <2 Hz, thiazole-H); 2.87 (3H-s: 2-CH₃); 2.32 (3H-d, J<2 Hz, 5′-CH₃):2 exchangeable protons.

R_{F}=0.11 (Merck ready-made TLC plates, silica gel 60 F-254; eluant:dichloromethane/methanol 95:5 v/v); 0.15 (eluant:chloroform/cyclohexane/methanol/conc. ammonia 68:15:15:2).

### EXAMPLE 2

### 4-hydroxy-2-methyl-N-(5-methyl-2-thiazolyl)-6-(trifluoromethyl)-2H-thieno[2,3-e]-1,2-thiazine-3-carboxamide-1,1-dioxide

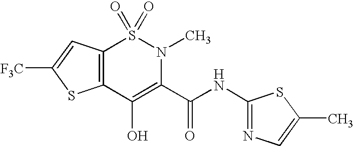

Prepared analogously to Example 1 from methyl 4-hydroxy-2-methyl-6-(trifluoromethyl)-2H-thieno[2,3-e]-1,2-thiazin-3-carboxylate-1,1-dioxide and 5-methyl-2-thiazolamine.

Yield: 58% of theory

R_{F}=0.13 (Merck ready-made TLC plates, silica gel 60 F-254; eluant:dichloromethane/methanol 95:5 v/v)

### EXAMPLE 3

### 6-bromo-4-hydroxy-2-methyl-N-(5-methyl-2-thiazolyl)-2H-thieno[2,3-e]-1,2-thiazine-3-carboxamide-1,1-dioxide

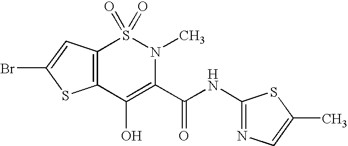

Prepared analogously to Example 1 from methyl 6-bromo-4-hydroxy-2-methyl-2H-thieno[2,3-e]-1,2-thiazin-3-carboxylate-1,1-dioxide and 5-methyl-2-thiazolamine.

Yield: 65 % of theory (yellowish-orange crystals)

Elemental analysis: C₁₂H₁₀BrN₃O₄S₃ (436.31)

| | | | | | |
|---|---|---|---|---|---|
| Calc.: | C 33.03 | H 2.31 | Br 18.31 | N 9.63 | S 22.04 |
| Found: | C 33.20 | H 2.42 | Br 18.06 | N 9.90 | S 21.85 |

IR (KBr): C═O 1615, amide-II 1540, SO₂ 1340, 1175/cm.

R_{F}=0.18 (Merck ready-made TLC plates, silica gel 60 F-254; eluant chloroform/-cyclohexane/methanol/conc. ammonia 68:15:15:2)

The Examples that follow describe the preparation of some pharmaceutical formulations:

### EXAMPLE I

### Tablets containing 10 mg 6-chloro-4-hydroxy-2-methyl-N-(5-methyl-2-thiazolyl)-2H-thieno[2,3-e]-1,2-thiazine-3-carboxamide-1,1-dioxide

Composition:

1 tablet contains:

| | | |
|---|---|---|
| active substance | 10.0 | mg |
| maize starch | 112.0 | mg |
| polyvinylpyrrolidone | 175.0 | mg |
| magnesium stearate | 3.0 | mg |
| | 300.0 | mg |

Method of Preparation:

The mixture of the active substance with maize starch is granulated with a 14% solution of the polyvinylpyrrolidone in water through a 1.5 mm mesh screen, dried at 45° C. and again passed through the same screen. The granules thus obtained are mixed with magnesium stearate and compressed into tablets.

| | | |
|---|---|---|
| Weight of tablet: | 300 | mg |
| Punch: | 10 | mm, flat |

### EXAMPLE II

### Coated tablets containing 10 mg of 6-chloro-4-hydroxy-2-methyl-N-(5-methyl-2-thiazolyl)-2H-thieno[2,3-e]-1,2-thiazine-3-carboxamide-1,1-dioxide

Composition:

1 tablet core contains:

| | | |
|---|---|---|
| active substance | 10.0 | mg |
| maize starch | 260.0 | mg |
| gelatine | 8.0 | mg |
| talc | 18.0 | mg |
| magnesium stearate | 4.0 | mg |
| | 300.0 | mg |

Method of Preparation:

The mixture of the active substance with maize starch is granulated with a 10% aqueous gelatine mixture through a 1.5 mm mesh screen, dried at 45° C. and again passed through the same screen. The granules thus obtained are mixed with talc and magnesium stearate and compressed into tablet cores.

| | | |
|---|---|---|
| Weight of core: | 300 | mg |
| Punch: | 10 | mm, convex |

The tablet cores are coated by known methods with a shell consisting essentially of sugar and talc. The finished coated tablets are polished with beeswax.

| | |
|---|---|
| Weight of coated tablet: | 540 mg |

### EXAMPLE III

### Gelatine capsules containing 10 mg of 6-chloro-4-hydroxy-2-methyl-N-(5-methyl-2-thiazolyl)-2H-thieno[2,3-e]-1,2-thiazine-3-carboxamide-1,1-dioxide

Composition:

1 gelatine capsule contains:

| | | |
|---|---|---|
| active substance | 10.0 | mg |
| maize starch | 400.0 | mg |
| Aerosil | 6.0 | mg |
| magnesium stearate | 8.0 | mg |
| | 424.0 | mg |

Method of Preparation:

The substances are intensively mixed and packed into size 1 gelatine capsules. Capsule contents: 424 mg

### EXAMPLE IV

### Suppositories containing 15 mg of 6-chloro-4-hydroxy-2-methyl-N-(5-methyl-2-thiazolyl)-2H-thieno[2,3-e]-1,2-thiazine-3-carboxamide-1,1-dioxide

Composition:

1 suppository contains:

| | | |
|---|---|---|
| active substance | 15.0 | mg |
| suppository mass (e.g. Witepsol W 45) | 1725.0 | mg |
| | 1740.0 | mg |

Method of Preparation:

The finely powdered active substance is stirred using an immersion homogeniser into the molten suppository mass which has been cooled to 40° C. The mass is poured at 38° C. into slightly chilled moulds.

What is claimed is:

1. A 4-Hydroxy-2H-thieno[2,3-e]-1,2-thiazine-3-carboxamide-1,1-dioxide of formula 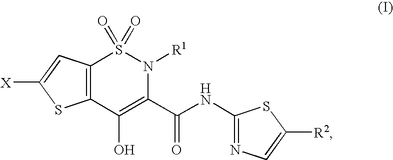
wherein
X denotes a fluorine, chlorine or bromine atom or the trifluoromethyl group,
R¹ denotes a hydrogen atom or a methyl or ethyl group and
R² denotes a methyl or ethyl group, or
a physiologically acceptable salt thereof with an inorganic or organic base.

2. 6-Chloro-4-hydroxy-2-methyl-N-(5-methyl-2-thiazolyl )-2H-thieno[2,3-e]-1,2-thiazine-3-carboxamide-1,1-dioxide, or a physiologically acceptable salt thereof with an inorganic or organic base.

3. A pharmaceutical composition of matter comprising a compound according to claim 1 with one or more carriers and/or excipients.

4. A method for treating inflammation in a warm-blooded animal which comprises administering to said animal a therapeutically effective amount of a compound according to claim 1.

5. A method for treating acute episodes of intermittent and chronic activated arthrosis in a warm-blooded animal which comprises administering to said animal a therapeutically effective amount of a compound according to claim 1.

6. A method for treating rheumatoid arthritis (chronic polyarthritis) in a warm-blooded animal which comprises administering to said animal a therapeutically effective amount of a compound according to claim 1.

7. A method for treating neoplasias in a warm-blooded animal which comprises administering to said animal a therapeutically effective amount of a compound according to claim 1.

8. A method for treating acute and persistent pain in a warm-blooded animal which comprises administering to said animal a therapeutically effective amount of a compound according to claim 1.
